(19) United States
(12) Patent Application Publication
(10) Pub. No.: US 2008/0275011 A1
(43) Pub. Date: Nov. 6, 2008

(54) USE OF DIPYRIDAMOLE, ACETYLSALICYLIC ACID AND AN ANGIOTENSIN II ANTAGONIST FOR TREATMENT AND PREVENTION OF VASCULAR EVENTS

(76) Inventors: James C. Gilbert, Bethlehem, CT (US); David Michael Humphreys, Anghiari (IT); Lutz Hilbrich, Mainz-Kastel (DE); Axel Riedel, Maselheim (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 12/175,510

(22) Filed: Jul. 18, 2008

Related U.S. Application Data
(63) Continuation of application No. 10/912,966, filed on Aug. 6, 2004, now abandoned.
(60) Provisional application No. 60/493,842, filed on Aug. 8, 2003.

(51) Int. Cl. A61K 31/60; A61P 9/10

(52) U.S. Cl. 514/161

(57) ABSTRACT

This invention relates to a method of treating and preventing vascular events and circulatory disorders in a patient in need thereof, especially in a patient at risk for said indications, using dipyridamole in combination with acetylsalicylic acid (ASA) and an angiotensin II antagonist, corresponding pharmaceutical compositions, and the use of dipyridamole for the manufacture of a corresponding pharmaceutical composition comprising a combination of dipyridamole, acetyl salicylic acid and an angiotensin II antagonist.

### RELATED APPLICATIONS

This application is a continuation of U.S. Ser. No. 10/912,966, filed Aug. 6, 2004, which claims the benefit to U.S. Provisional Application Ser. No. 60/493,842, filed Aug. 8, 2003.

### FIELD OF THE INVENTION

This invention relates to a method of treatment and prevention of indications (A) selected from
acute myocardial infarction, secondary myocardial infarction and cardiovascular death,

in a person in need thereof, especially in persons having elevated risk of cardiovascular events, e.g. in hypertensive patients,

a method of treatment and prevention of indications (B) selected from
pulmonary embolism, retinal vascular accident, deep vein thrombosis, peripheral arterial occlusion, transient ischemic attack (TIA), ischaemic peripheral circulatory disorders, myocardial ischaemia (angina), thrombotic thrombocytopenic purpura, and progression of cardiac insufficiency after myocardial infarction,

in a patient in need thereof, especially in persons having generally elevated risk of vascular events including arterial, venous, central and peripheral vascular events, e.g. in diabetic, obese and hypertensive patients,

a method of preventing or treating indications (C), which can be positively influenced by organoprotective, tissue-protective and vasculoprotective effects of the drugs used, selected from
renoprotection, e.g. in renal failure or diabetic nephropathy,
left ventricular hypertrophy, vascular thickening, e.g. prevention of thickening of blood vessel walls after vascular operations, prevention of arterial restenosis after angioplasty, prevention or treatment of atherosclerosis, prevention of diabetic angiopathy and diabetic retinopathy, and

a method of preventing or treating indications (D) selected from
obstructive airways diseases, chronic obstructive pulmonary disease, e.g. bronchitis or chronic bronchitis, emphysema, likewise from asthma, cystic fibrosis, interstitial lung disease, lung cancer, pulmonary vascular disease, and increased resistance to airflow during forced expiration,
adult respiratory distress syndrome (ARDS), reducing the proliferative capacity of the epithelium in lung and breast cancer, the treatment of sepsis syndrome, lung injury forms, such as pneumonia aspiration of gastric content, chest trauma, shock, burns, fat embolia, cardiopulmonary bypass, O₂ toxicity, haemorrhagic pancreatitis, interstitial and bronchoalveolar inflammation, proliferation of epithelial and interstitial cells, collagen accumulation or fibrosis,

which methods comprise co-administration of effective amounts of dipyridamole (DIP) in combination with acetylsalicylic acid (ASA) and an angiotensin (ANG) II antagonist to a person in need of such treatment,

suitable pharmaceutical compositions comprising DIP in combination with ASA and an ANG II antagonist as a combined preparation for simultaneous, separate or sequential use in treatment of said indications and

the use of an ANG II antagonist for manufacture of a pharmaceutical composition for treatment of said indications when used in combination with DIP in combination with ASA.

The method of preventing indications mentioned hereinbefore should be understood to reduce the risk for said indications or to reduce the incidence of said indications in patient populations having elevated risk for said indications.

### BACKGROUND OF THE INVENTION

Dipyridamole {2,6-bis(diethanolamino)-4,8-dipiperidino-pyrimido[5,4-d]pyrimidine}, closely related substituted pyrimido-pyrimidines and their preparation have been described in e.g. U.S. Pat. No. 3,031,450. DIP was introduced as a coronary vasodilator in the early 1960s. It is also well known having platelet aggregation inhibitor properties due to the inhibition of adenosine uptake. Subsequently, DIP was shown to reduce thrombus formation in a study of arterial circulation of the brain in a rabbit model. These investigations led to its use as an anti-thrombotic agent; it soon became the therapy of choice for such applications as stroke prevention, maintaining the patency of coronary bypass and valve-replacement, as well as for treatment prior to coronary angioplasty.

Furthermore, the European Stroke Prevention Study 2 (ESPS-2; J Neurol Sci. 1996; 143: 1-13; Neurology 1998; 51: 17-19) proved that treatment by DIP alone was as effective as low-dose aspirin in the reduction of stroke risk, and combination therapy with DIP and aspirin was more than twice as effective as aspirin alone.

DIP appears to inhibit thrombosis through multiple mechanisms. Early studies showed that it inhibits the uptake of adenosine, which was found to be a potent endogenous anti-thrombotic compound. DIP was also shown to inhibit cyclic AMP phosphodiesterase, thereby increasing intracellular c-AMP.

DIP also has antioxidant properties (Free Radic. Biol. Med. 1995; 18: 239-247) that may contribute to its anti-thrombotic effect. When oxidized, low density lipoproteins become recognized by the scavenger receptor on macrophages, which is assumed to be the necessary step in the development of atherosclerosis (Ann. Rev. Med. 1992; 43: 219-25).

The inhibition of free radical formation by DIP has been found to inhibit fibrinogenesis in experimental liver fibrosis (Hepatology 1996; 24: 855-864) and to suppress oxygen radicals and proteinuria in experimental animals with aminonucleoside nephropathy (Eur. J. Clin. Invest. 1998; 28: 877-883; Renal Physiol. 1984; 7: 218-226). Inhibition of lipid peroxidation also has been observed in human nonneoplastic lung tissue (Gen. Pharmacol. 1996; 27: 855-859).

ANG II plays a major role in pathophysiology, especially as the most potent blood pressure increasing agent in humans. ANG II antagonists therefore are suitable for treating elevated blood pressure and congestive heart failure in a mammal. Examples of ANG II antagonists are described in EP-A-0 502 314, EP-A-0 253 310, EP-A-0 323 841, EP-A-0 324 377, U.S. Pat. No. 4,355,040 and U.S. Pat. No. 4,880,804. Specific ANG II antagonists are sartans such as candesartan, eprosartan, irbesartan, losartan, telmisartan or valsartan, furthermore olmesartan and tasosartan.

It is known that ANG II, besides its blood pressure increasing effect, additionally features growth promoting effects contributing to left ventricular hypertrophy, vascular thickening, atherosclerosis, renal failure and stroke. Bradykinin, on the other hand, exerts vasodilating and tissue protective actions, as disclosed for instance by W. Wienen et al.: Antihypertensive and renoprotective effects of telmisartan after long term treatment in hypertensive diabetic (D) rats, 2nd. Int. Symposium on Angiotensin II Antagonism, Feb. 15-18, 1999, The Queen Elizabeth II Conference Center, London, UK, Book of Abstracts, Abstract No. 50.

Losartan and irbesartan provide a renoprotective effect found within first clinical trials, as disclosed for instance by S. Andersen et al.: Renoprotective effects of angiotensin II receptor blockade in type 1 diabetic patients with diabetic nephropathy, Kidney Int 57 (2), 601-606 (2000).

ANG II antagonists selectively block the AT₁ receptor, leaving the AT₂ receptor, which plays a role in anti-growth and tissue regenerative actions, unopposed. Completed clinical trials with ANG II antagonists appear to display similar blood pressure reducing and tissue protective effects as with ACE inhibitors, as disclosed for instance by D. H. G. Smith et al.: Once-daily telmisartan compared with enalapril in the treatment of hypertension, Adv. Ther 1998, 15: 229-240, and by B. E. Karlberg et al.: Efficacy and safety of telmisartan, a selective AT1 receptor antagonist, compared with enalapril in elderly patients with primary hypertension, J Hypertens 1999, 17: 293-302.

EP-A-1 013273 discloses the use of AT₁ receptor antagonists or AT₂ receptor modulators for treating diseases associated with an increase of AT₁ receptors in subepithelial area or increase of AT₂ receptors in the epithelia, especially for treatment of several lung diseases.

### SUMMARY OF THE INVENTION

Surprisingly it has been found that administration of DIP in combination with ASA and an ANG II antagonist provides unexpected efficacy in the prevention or treatment of indications (A) to (D) mentioned hereinbefore.

ASA inhibits aggregation through direct effects on the platelet, in more detail, by irreversibly acetylating platelet cyclooxygenase, thus inhibiting the production of thromboxane, which is strongly thrombotic. In high doses, however, aspirin crosses over into endothelial cells (N. Eng. J. Med. 1984; 311: 1206-1211), where it interrupts the production of prostacyclin, a potent natural inhibitor of platelet aggregation and by-product of the “arachidonic cascade” (N. Engl. J. Med. 1979; 300: 1142-1147). These observations led to the concept of low-dose antiplatelet therapy with ASA to maximize inhibition of thromboxane while minimizing the loss of prostacyclin (Lancet 1981; 1: 969-971). In the method of prevention according to the invention a combination of low-dose ASA with DIP and an ANG II antagonist is preferred.

Viewed from one aspect the present invention provides a method for preventing or treating indications (A) selected from
acute myocardial infarction, secondary myocardial infarction and cardiovascular death,

in a person in need thereof, especially in persons having elevated risk of cardiovascular events, e.g. in hypertensive patients,

a method of preventing or treating indications (B) selected from
pulmonary embolism, retinal vascular accident, deep vein thrombosis, peripheral arterial occlusion, transient ischemic attack (TIA), ischaemic peripheral circulatory disorders, myocardial ischaemia (angina), thrombotic thrombocytopenic purpura, and progression of cardiac insufficiency after myocardial infarction,

in a patient in need thereof, especially in persons having generally elevated risk of vascular events including arterial, venous, central and peripheral vascular events, e.g. in diabetic, obese and hypertensive patients,

a method of preventing or treating indications (C), which can be positively influenced by organoprotective, tissue-protective and vasculoprotective effects of the drugs used, selected from
renoprotection, e.g. in renal failure or diabetic nephropathy,
left ventricular hypertrophy, vascular thickening, e.g. prevention of thickening of blood vessel walls after vascular operations, prevention of arterial restenosis after angioplasty, prevention or treatment of atherosclerosis, prevention of diabetic angiopathy and diabetic retinopathy, and

a method of preventing or treating indications (D) selected from
obstructive airways diseases, chronic obstructive pulmonary disease, e.g. bronchitis or chronic bronchitis, emphysema, likewise from asthma, cystic fibrosis, interstitial lung disease, lung cancer, pulmonary vascular disease, and increased resistance to airflow during forced expiration, adult respiratory distress syndrome (ARDS), reducing the proliferative capacity of the epithelium in lung and breast cancer, the treatment of sepsis syndrome, lung injury forms, such as pneumonia aspiration of gastric content, chest trauma, shock, burns, fat embolia, cardiopulmonary bypass, O₂ toxicity, haemorrhagic pancreatitis, interstitial and bronchoalveolar inflammation, proliferation of epithelial and interstitial cells, collagen accumulation or fibrosis,

said method comprising administering to a patient in need of such treatment an effective amount of a pharmaceutical composition comprising DIP or a pharmaceutically acceptable salt thereof in combination with ASA and an ANG II antagonist.

Viewed from a second aspect the present invention provides a pharmaceutical composition comprising DIP or a pharmaceutically acceptable salt thereof in combination with ASA and an ANG II antagonist, adapted for simultaneous, separate or sequential administration.

The pharmaceutical composition according to the invention is meant to comprise a fixed dose combination comprising the active ingredients in one formulation together as well as

a kit of parts comprising
(a) a first containment containing a pharmaceutical composition comprising a therapeutically effective of DIP; and
(b) a second containment containing a pharmaceutical composition comprising ASA and a pharmaceutically acceptable carrier; and
(c) a third containment containing a pharmaceutical composition comprising an ANG II antagonist.

Viewed from a different aspect the present invention provides the use of DIP or a pharmaceutically acceptable salt thereof in combination with ASA and an ANG II antagonist for the manufacture of a pharmaceutical composition for prevention and/or treatment of indications (A) to (D) mentioned hereinbefore in a patient in need thereof.

### DETAILED DESCRIPTION OF THE INVENTION

The invention provides a new and improved approach for prevention and or treatment of indications (A) to (D) mentioned hereinbefore comprising administering to the patient an effective amount of a pharmaceutical composition containing as active ingredients DIP or a pharmaceutically acceptable salt thereof in combination with ASA and an ANG II antagonist.

In the method of the invention any of the oral DIP retard, instant or the parenteral formulations on the market may be used, the retard formulations being preferred, for instance those available under the trademark Persantin®, or, already in combination with ASA the formulations available under the trademark Asasantin® or Aggrenox®. Suitable DIP retard formulations are disclosed in EP-A-0032562, instant formulations are disclosed in EP-A-0068191 and combinations of ASA with DIP are disclosed in EP-A-0257344 which are incorporated by reference. Any ANG II antagonist known in the art may be used in the method of prevention of the invention, e.g. the sartans such as candesartan, eprosartan, irbesartan, losartan, telmisartan (trademark Micardis®), valsartan, olmesartan or tasosartan, including the salts thereof or polymorphs thereof, using for instance the dosages disclosed in Rote Liste® 2003, Editio Cantor Verlag Aulendorf, Germany, or in Physician's Desk Reference, 57 edition, 2003.

In the method of prevention according to the invention a plasma level of DIP of about 0.2 μmol/L to 5 μmol/L, preferably of about 0.5 μmol/L to 2 μmol/L or particularly of about 0.8 μmol/L to 1.5 μmol/L may be maintained. DIP can be administered orally in a daily dosage of 50 mg to 750 mg, preferably 100 mg to 500 mg, most preferred 200 mg to 450 mg, for instance 200 mg twice a day.

With respect to ASA this component of the ternary medication may be administered orally in a daily dosage of 10 mg to 200 mg, preferably 25 mg to 100 mg, most preferred 30 mg to 75 mg, for instance 25 mg twice a day.

With respect to the third component the ANG II antagonist telmisartan is preferred. This component can be administered orally in a daily dosage of 10 mg to 200 mg, for instance in a daily dosage of 20 mg, 40 mg, 80 mg or 160 mg, preferably in a daily dosage of 40 mg to 160 mg, most preferred 60 mg to 100 mg, for instance 20 mg or 40 mg once a day.

A specific method of prevention according to the invention comprises the combination of DIP administered orally 200 mg twice a day, ASA administered orally 25 mg twice a day and telmisartan administered orally 20 mg, 40 mg or 80 mg once a day.

With respect to all aspects of the invention the combination of DIP, ASA and telmisartan is preferred, especially in the oral dosages indicated hereinbefore as most preferred.

What is claimed is:

1. A method for preventing or treating indications (A) selected from
acute myocardial infarction, secondary myocardial infarction or cardiovascular death,
said method comprising administering to a patient in need of such treatment an effective amount of a pharmaceutical composition comprising dipyridamole or a pharmaceutically acceptable salt thereof in combination with acetylsalicylic acid and an angiotensin II antagonist.

2. A method for preventing or treating indications (B) selected from
pulmonary embolism, retinal vascular accident, deep vein thrombosis, peripheral arterial occlusion, transient ischemic attack (TIA), ischaemic peripheral circulatory disorders, myocardial ischaemia (angina), thrombotic thrombocytopenic purpura, or progression of cardiac insufficiency after myocardial infarction,
said method comprising administering to a patient in need of such treatment an effective amount of a pharmaceutical composition comprising dipyridamole or a pharmaceutically acceptable salt thereof in combination with acetylsalicylic acid and an angiotensin II antagonist.

3. A method for preventing or treating indications (C), which can be positively influenced by organoprotective, tissue-protective and vasculoprotective effects of the drugs used, selected from
renoprotection, left ventricular hypertrophy, or vascular thickening,
said method comprising administering to a patient in need of such treatment an effective amount of a pharmaceutical composition comprising dipyridamole or a pharmaceutically acceptable salt thereof in combination with acetylsalicylic acid and an angiotensin II antagonist.

4. A method for preventing or treating indications (D) selected from
obstructive airway diseases, chronic obstructive pulmonary disease, emphysema, asthma, cystic fibrosis, interstitial lung disease, lung cancer, pulmonary vascular disease, increased resistance to airflow during forced expiration, adult respiratory distress syndrome (ARDS), reduced proliferative capacity of the epithelium in lung and breast cancer, sepsis syndrome, lung injury, chest trauma, shock, burns, fat embolia, cardiopulmonary bypass, 02 toxicity, haemorhagic pancreatitis, interstitial and bronchoalveolar inflammation, proliferation of epithelial and interstitial cells, collagen accumulation or fibrosis,
said method comprising administering to a patient in need of such treatment an effective amount of a pharmaceutical composition comprising dipyridamole or a pharmaceutically acceptable salt thereof in combination with acetylsalicylic acid and an angiotensin II antagonist.

5. The method of claim 2 wherein the angiotensin II antagonist is telmisartan.

6. The method of claim 3 wherein the angiotensin II antagonist is telmisartan.

7. The method of claim 4 wherein the angiotensin II antagonist is telmisartan.

8. A pharmaceutical composition comprising dipyridamole or a pharmaceutically acceptable salt thereof, acetyl salicylic acid and an angiotensin II antagonist for simultaneous, separate or sequential use.

9. The pharmaceutical composition of claim 8 wherein the angiotensin II antagonist is telmisartan.
